(19) United States
(12) Patent Application Publication
(10) Pub. No.: US 2026/0250253 A1
(43) Pub. Date: Aug. 27, 2026

(54) METHOD FOR PRODUCING 2-[4-(4-CHLOROPHENOXY)-2-(TRIFLUOROMETHYL)PHENYL]-1-(1,2,4-TRIAZOL-1-YL)PROPAN-2-OL

(71) Applicant: BASF AGRO B.V., Arnhem (NL)

(72) Inventors: David A. Harney, Palmyra, MO (US); David A. Cortes, Palmyra, MO (US); Franklin Brice Caldwell, Troy, MO (US); Keith W. Rosenkrans, Palmyra, MO (US); Przemyslaw Mariusz Lusiak, Palmyra, MO (US); Ryan Michael Phillips, Palmyra, MO (US)

(21) Appl. No.: 18/875,806

(22) PCT Filed: Jun. 12, 2023
(86) PCT No.: PCT/EP2023/065629

§ 371 Date: Dec. 17, 2024

(30) Foreign Application Priority Data
Jun. 20, 2022 (EP) 22179797.0

(51) Int. Cl. C07D 249/08

(52) U.S. Cl. CPC C07D 249/08

(57) ABSTRACT

The present invention relates to a method for producing 2-[4-(4-chlorophenoxy)-2-(trifluoromethyl)phenyl]-1-(1,2,4-triazol-1-yl)propan-2-ol in high purity and yield.

The present invention relates to a method for producing 2-[4-(4-chlorophenoxy)-2-(trifluoromethyl)phenyl]-1-(1,2,4-triazol-1-yl)propan-2-ol in high purity and high yield. 2-[4-(4-Chlorophenoxy)-2-(trifluoromethyl)phenyl]-1-(1,2,4-triazol-1-yl)propan-2-ol is an effective fungicide described, for example, in WO 2014/108286 and WO 2015/091045. It is generally synthesized from 2-[4-(4-chlorophenoxy)-2-(trifluoromethyl)phenyl]-2-methyl-oxirane which is reacted with 1H-[1,2,4]-triazole. The reaction is, however, not perfectly selective. Apart from the desired 1,2,4-triazol-1-yl compound, the undesired 1,2,4-triazol-4-yl isomer is also formed.

In agricultural chemistry, it is essential to provide active desired 1,2,4-triazol-1-yl compound ingredients in high purity in order to ensure a high reliability of the product and avoid unexpected and undesired side effects stemming from side products or other impurities.

Furthermore, yield loss must be minimized.

WO 2017/102905 describes a method for producing 2-[4-(4-chlorophenoxy)-2-(trifluoromethyl)phenyl]-1-(1,2,4-triazol-1-yl)propan-2-ol in high purity. According to this method the product is purified by crystallization. The crystallisation, however, leads to some product being lost to the filtrate. So, the filtrate contains both the desired 1,2,4-triazol-1-yl compound and a nearly all the formed undesired 1,2,4-triazol-4-yl isomer.

Surprisingly we have found that the filtrate can be purified from the undesired 1,2,4-triazol-4-yl isomer by precipitating said isomer and then separating it from the mother liquid.

Thereafter, the mother liquid enriched in the desired product is returned back into the process. In this manner, more than 90% of the product lost to the mother liquid after crystallization can be recovered and re-introduced into the process while maintaining the needed high purity of the final product. Moreover, in this way the amount of aromatic solvents used for crystallization can be reduced.

It was therefore the object of the present invention to provide a method which yields 2-[4-(4-chlorophenoxy)-2-(trifluoromethyl)phenyl]-1-(1,2,4-triazol-1-yl)propan-2-ol in high purity, minimizes the loss of the desired 1,2,4-triazol-1-yl compound during purification and reduces the amount of aromatic solvents used in the process.

Hence, the present invention relates to a method for obtaining 2-[4-(4-chlorophenoxy)-2-(trifluoromethyl)phenyl]-1-(1,2,4-triazol-1-yl)propan-2-ol of formula (I) from a mixture containing the compound of formula (I) and 2-[4-(4-chlorophenoxy)-2-(trifluoromethyl)phenyl]-1-(1,2,4-triazol-4-yl)propan-2-ol of formula (I-sym)

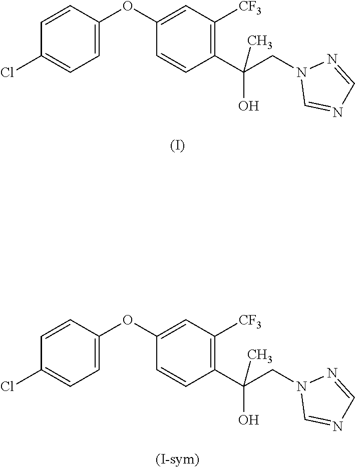 [SMILES: CC(O)(Cn1cncn1)c1ccc(Oc2ccc(Cl)cc2)cc1C(F)(F)F ; "(I)" ; CC(O)(Cn1cncn1)c1ccc(Oc2ccc(Cl)cc2)cc1C(F)(F)F ; "(I-sym)"]

which method comprises
(a) providing a mixture containing the compounds of formulae (I) and (I-sym) in at least one aromatic solvent, where the mixture contains the compound of formula (I) in a concentration of from 30 to 90% by weight, relative to the total weight of the mixture;
(b) adding to the mixture of step (a) at least one polar aprotic solvent, so that it is contained in an amount of from 1 to 25% by weight, relative to the weight of the solution obtained after the addition of the at least one polar aprotic solvent;
(c) crystallizing the compound of formula (I) from the mixture obtained in step (b);
(d) adding to the mother liquid obtained after crystallization in step (c) water in an amount of from 10 to 40% by weight, relative to the total weight of the mother liquid; and
(e) separating the liquid phase from the mixture obtained in step (d) and returning the separated liquid phase to step (a).

Mixtures containing compounds (1) and (I-sym) are generally formed when the compound (I) is prepared by nucleophilic addition of or substitution by 1H-[1,2,4]-triazole to/at an electrophilic carbon atom of a precursor molecule of compound (I). For instance, such mixtures are generally formed when 2-[4-(4-chlorophenoxy)-2-(trifluoromethyl)phenyl]-2-methyl-oxirane is reacted with 1H-[1,2,4]-triazole.

Therefore, step (a), for example, may comprise the following steps:
(i) reacting 2-[4-(4-chlorophenoxy)-2-(trifluoromethyl)phenyl]-2-methyl-oxirane of formula (II)

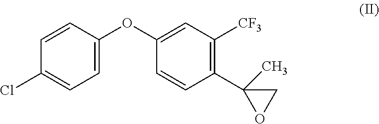 [SMILES: CC1(c2ccc(Oc3ccc(Cl)cc3)cc2C(F)(F)F)CO1]

with 1H-[1,2,4]-triazole in the presence of a base in at least one polar aprotic solvent;
(ii) after completion of the reaction removing at least 90% of the at least one polar aprotic solvent;
(iii) diluting the mixture obtained in step (ii) with at least one aromatic solvent and extracting the diluted mixture with water or an aqueous solution;
(iv) adjusting the amount of the aromatic solvent in the organic phase obtained in step (iii) to such an extent that the resulting mixture contains the compound (I) in a concentration of from 30 to 90% by weight, relative to the total weight of the mixture.

Hence, the present invention, in one aspect, relates to a method for obtaining 2-[4-(4-chlorophenoxy)-2-(trifluoromethyl)phenyl]-1-(1,2,4-triazol-1-yl)propan-2-ol of formula (I) from a mixture containing the compound of formula (I) and 2-[4-(4-chlorophenoxy)-2-(trifluoromethyl)phenyl]-1-(1,2,4-triazol-4-yl)propan-2-ol of formula (I-sym)

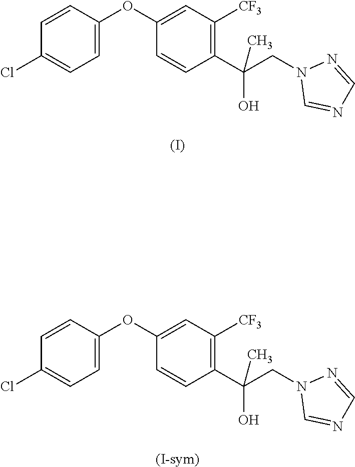 [SMILES: CC(O)(Cn1cncn1)c1ccc(Oc2ccc(Cl)cc2)cc1C(F)(F)F ; "(I)" ; CC(O)(Cn1cncn1)c1ccc(Oc2ccc(Cl)cc2)cc1C(F)(F)F ; "(I-sym)"]

which method comprises
(a) providing a mixture containing the compounds of formulae (I) and (I-sym) in at least one aromatic solvent, where the mixture contains the compound of formula (I) in a concentration of from 30 to 90% by weight, relative to the total weight of the mixture, wherein the mixture is provided by
(i) reacting 2-[4-(4-chlorophenoxy)-2-(trifluoromethyl)phenyl]-2-methyl-oxirane of formula (II)

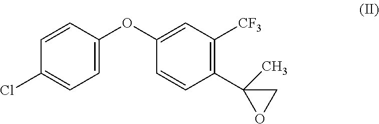 [SMILES: CC1(c2ccc(Oc3ccc(Cl)cc3)cc2C(F)(F)F)CO1]

with 1H-[1,2,4]-triazole in the presence of a base in at least one polar aprotic solvent;
(ii) after completion of the reaction removing at least 90% of the at least one polar aprotic solvent;
(iii) diluting the mixture obtained in step (ii) with at least one aromatic solvent and extracting the diluted mixture with water or an aqueous solution;
(iv) adjusting the amount of the aromatic solvent in the organic phase obtained in step (iii) to such an extent that the resulting mixture contains the compound (I) in a concentration of from 30 to 90% by weight, relative to the total weight of the mixture.
(b) adding to the mixture of step (a) at least one polar aprotic solvent, so that it is contained in an amount of from 1 to 25% by weight, relative to the weight of the solution obtained after the addition of the at least one polar aprotic solvent;
(c) crystallizing the compound of formula (I) from the mixture obtained in step (b);
(d) adding to the mother liquid obtained after crystallization in step (c) water in an amount of from 10 to 40% by weight, relative to the total weight of the mother liquid; and
(e) separating the liquid phase from the mixture obtained in step (d) and returning the separated liquid phase to step (a)(iii).

The mixture provided in step (a) may comprise, apart from compounds (1) and (I-sym) and the at least one aromatic solvent, impurities, such as unreacted starting material from which compounds (1) and (I-sym) are formed. They may also comprise traces of other organic solvents (i.e. solvents different from the aromatic solvent(s) used in step (a)). These impurities and other organic solvents, if present, are however contained in minor amounts, preferably in an overall amount of at most 15% by weight, more preferably at most 10% by weight, in particular at most 5% by weight, relative to the overall weight of the mixture. Especially the other organic solvents, if present, are contained in an amount of at most 10% by weight, preferably at most 8% by weight, in particular at most 5% by weight, specifically at most 2% by weight, relative to the overall weight of the mixture.

In step (a)(i) 1H-[1,2,4]-triazole is preferably used in at least equimolar amounts with respect to the oxirane (II). Preferably, the oxirane (II) and 1H-[1,2,4]-triazole are used in a molar ratio of from 1:1 to 1:4, more preferably from 1:1 to 1:3, in particular from 1:1.1 to 1:2, more particularly from 1:1.1 to 1:1.5 and specifically from 1:1.1 to 1:1.35.

The base used in step (a)(i) can be an inorganic or organic base. Inorganic bases are for example alkali and earth alkaline hydroxides, carbonates, hydrogen carbonates, phosphates and hydrogen phosphates. Organic bases are for example pyridine, substituted pyridines, like lutidine or 4-(dimethylamino)-pyridine, tertiary amines, like triethylamine, tripropylamine, diisopropylethylamine or morpholine, or cyclic amidines, like 1,4-diazabicyclo[2.2.2]octan (DABCO), 1,8-diazabicyclo[5.4.0]undec-7-ene (DBU) or 1,5-diazabicyclo(4.3.0)non-5-ene (DBN).

Preferably, however, inorganic bases are used. Preferred inorganic bases are alkali metal hydroxides, carbonates and phosphates, especially LiOH, NaOH, KOH, Li₂CO₃, Na₂CO₃, K₂CO₃, Cs₂CO₃, Li₃PO₄, Na₃PO₄ or K₃PO₄. In particular alkali metal hydroxides, and especially NaOH or KOH are used. Very specifically NaOH is used.

In a specific embodiment at least a part of 1H-[1,2,4]-triazole is used as the corresponding alkali metal salt, e.g. as its Li⁺, Na⁺ or K⁺ salt. This is obtained by reacting 1H-[1,2,4]-triazole with the respective hydroxide (i.e. LiOH, NaOH or KOH), hydride (LiH or NaH) or alcoholate (e.g. sodium or potassium methanolate, ethanolate or tert-butanolate) and isolating it before introducing it into the reaction of step (a)(i).

Strong bases, i.e. bases with a pK_{b} below 3.75, are preferably used in at most stoichiometric amounts with respect to the oxirane (II). In case of bases derived from polybasic acids, such as the carbonates and phosphates, and also in case of bases derived from monobasic acids, but with a two or three times charged counter cation, such as earth alkaline hydroxides, e.g. Ca(OH)₂, the term “stoichiometric” takes of course into consideration how many protons the base can neutralize. For instance, in case of an alkali metal hydroxide, stoichiometric amounts with respect to oxirane (II) means a molar ratio of 1:1, while in case of alkali or earth alkaline metal carbonates or of earth alkaline metal hydroxides stoichiometric amounts with respect to oxirane (II) mean a molar ratio of oxirane to base of 2:1, and in case of alkali metal phosphates stoichiometric amounts with respect to oxirane (II) mean a molar ratio of oxirane to phosphate of 3:1.

Weaker bases (i.e. bases with a pK_{b} of at least 3.75) are preferably used in at least stoichiometric amounts. For “stoichiometric amounts” in case of bases derived from polybasic acids the above remarks apply.

In case of bases derived from strong monobasic acids and with a monovalent counter cation, here the alkali metal hydroxides, the base is preferably used in an amount of from 0.2 to 1 mol per mol of oxirane (II), in particular from 0.3 to 0.7 mol per mol of oxirane (II).

Step (a)(i) is generally carried out at from 100° C. to the boiling point of the reaction mixture. It has however been found that the compound of formula (I) is obtained in higher purity if the reaction in step (a)(i) is carried out at from 110 to 130° C., in particular from 110 to 120° C. and specifically from 110 to 115° C.

Aprotic solvents are solvents without a functional group from which a proton can dissociate. Polar solvents are solvents with a dielectric constant of greater than 15. Polar aprotic solvents combine both properties. Examples for polar aprotic solvents are amides, such as N,N-dimethylformamide (DMF) and N,N-dimethylacetamide; sulfoxides, such as dimethylsulfoxide (DMSO); lactams, such as N-methylpyrrolidone (NMP); cyclic ethers, such as tetrahydrofuran, 1,3-dioxane and 1,4-dioxane; ketones such as acetone and methylethylketone; nitriles, such as acetonitrile; lactones, such as γ-butyrolactone; nitro compounds, such as nitromethane; ureas, such as tetramethyl urea or dimethylpropylene urea (DMPU), sulfones, such as sulfolan, and carbonic acids, such as dimethylcarbonate or ethylenecarbonate.

The at least one polar aprotic solvent used in step (a)(i) and (b) can be the same or different. It is independently selected from N,N-dimethylformamide, N,N-dimethylacetamide, dimethylsulfoxide, N-methylpyrrolidone, tetrahydrofuran, 1,4-dioxane, acetone, methylethylketone, acetonitrile and mixtures thereof; preferably, it is independently selected from N,N-dimethylformamide, N,N-dimethylacetamide, dimethylsulfoxide and N-methylpyrrolidone; more preferably, it is N,N-dimethylformamide.

In step (a)(ii), after completion of the reaction, all or at least almost all of the polar aprotic solvent(s), i.e. at least 90%, preferably at least 92%, in particular at least 95% and specifically at least 98% of the polar aprotic solvent(s) are removed. The percentages relate to the amount of solvent(s) introduced in step (a)(i) (which is considered as 100%).

Preferably the polar aprotic solvent(s) is/are removed to such an extent that in the resulting mixture the weight ratio of remaining polar aprotic solvent(s) to theoretically present compound (I) (i.e. if the yield is 100%) is at most 1:3, i.e. from 1:3 to 0:1, more preferably at most 1:4, i.e. from 1:4 to 0:1, in particular at most 1:7, i.e. from 1:7 to 0:1, more particularly at most 1:9, i.e. from 1:9 to 0:1, even more particularly at most 1:19, i.e. from 1:19 to 0:1, and specifically at most 1:24, i.e. from 1:24 to 0:1. The solvent(s) is/are generally removed by distillation.

The temperature used for distillation does preferably not exceed the reaction temperature of step (a)(i). Thus, distillation is preferably carried out under reduced pressure. The amount of removed solvent(s) can be determined, for example, by weighing the amount of removed solvent(s) and comparing it to the amount introduced in step (a)(i), or by gas chromatography of the reaction mixture obtained after removal of the solvent(s).

“Completion of the reaction” means that the reaction is carried out until no oxirane (II) can be detected anymore. Detection can be accomplished by standard methods, such as TLC, GC, HPLC or NMR of a sample of the reaction mixture.

In step (a)(iii) the mixture obtained in step (a)(ii) is diluted with at least one aromatic solvent and the diluted mixture is extracted with an aqueous medium, i.e. with water or an aqueous solution. Aqueous extraction is carried out in order to remove any salts formed, if present, excess base, if present, excess 1H-[1,2,4]-triazole, if present, remainders of the polar aprotic solvent(s), if present, and any other water-soluble components, if present. The at least one aromatic solvent can be added first and water or an aqueous solution can be added subsequently to the mixture obtained in step (a)(ii), or inversely, or the at least one aromatic solvent and water or the aqueous solution can be added simultaneously.

The at least one aromatic solvent used in step (a)(iii) is preferably selected from benzene, toluene, the xylenes and mixtures thereof; and is in particular toluene.

The aqueous solution is for example brine (saturated aqueous sodium chloride solution), non-saturated aqueous sodium chloride solution, or an acidic solution, e.g. diluted aqueous HCl.

Preferably the extracting agent is water or brine and is in particular water.

In a preferred embodiment, the extraction in step (a)(iii) is carried out at from 50 to 90° C., in particular from 70 to 90° C. Heating is mainly carried out in order to improve dissolution of the components of the mixture of step (a)(ii) in one of the two solvent systems (aromatic solvent or aqueous medium).

After extraction, i.e. after bringing the organic phase and the aqueous phase into close contact with each other, the aqueous phase and the organic phase are separated.

In step (a)(iv), if necessary, a part of the aromatic solvent(s) introduced in step (a)(iii) is removed from the organic phase obtained in step (a)(iii) to such an extent that the resulting mixture contains the compound (I) in a concentration of from 30 to 90% by weight, relative to the total weight of the resulting mixture (i.e. the mixture obtained after the removal of the aromatic solvent(s)).

Step (a)(iv) is necessary if the amount of aromatic solvent(s) introduced in step (a)(iii) is so high that compound (I) is contained in the mixture obtained after step (a)(iii) in a concentration below 30% by weight, relative to the total weight of the mixture.

Preferably, the at least one aromatic compound is removed to such an extent that the resulting mixture contains the compound (I) in a concentration of from 40 to 65% by weight, relative to the total weight of the mixture.

The concentration of the compound (I) in the mixture is determined by standard procedures, e.g. by gas chromatography or HPLC.

Analogously, the mixture of step (a) preferably contains the compound (I) in a concentration of from 40 to 65% by weight, relative to the total weight of the mixture.

In step (b) the at least one polar aprotic solvent is added to the mixture of step (a), in such an amount that it is contained in 1 to 25% by weight, preferably in 2 to 15% by weight, more preferably in 2 to 12% by weight, in particular in 2 to 9% by weight, specifically in 2 to 8% by weight and very specifically in 3 to 8% by weight, relative to the weight of the mixture obtained after its addition.

If the mixture of step (a) contains solids visible with the naked eye, this mixture is preferably heated before the least one polar aprotic solvent is added to obtain a mixture in which no solids are visible. Preferably, the mixture is heated to 50 to 100° C., in particular to 70 to 90° C. before the least one polar aprotic solvent is added.

Crystallization in step (c) is carried out by known methods, e.g. by simply allowing the mixture to stand; or by cooling the mixture, especially if it was heated before; or by heating and cooling again, or by reheating the cooled mixture and cooling it again. Cooling and heating can be repeated several times. Seed crystals of the compound of formula (I) can be added to the cooled solution in order to set off crystallization.

In particular, crystallization in step (c) is performed by cooling the preheated mixture of step (b) and optionally adding seed crystals; or, alternatively, by cooling the preheated mixture of step (b), reheating and cooling again.

Cooling means generally cooling to room temperature or below, preferably to +10° C. to −10° C., in particular to +5° C. to −5° C. and specifically to 0° C. Cooling is generally carried out within 1 to 12 h, preferably within 2 to 10 h, in particular within 6 to 10 h. Cooling can be carried out continually or stepwise, i.e. in several temperature steps.

The formed crystals are recovered from the mother liquid by standard methods, e.g. by filtration, sedimentation, decantation or centrifugation. If desired, the crystals are freed from remainders of the solvent, e.g. by washing with water and/or evaporation, especially under vacuum.

In step (d) the amount of water added to the mother liquid obtained in step (c) is usually from 10 to 40% by weight, relative to the total weight of the mother liquid.

In a preferred embodiment, the water is added to the mother liquid gradually, e.g. dropwise or in several portions. Preferably the addition of water is carried out over a period of 0.5 h to 5 h, more preferably over a period of 1 h to 4.5 h, most preferably over a period of 2 h to 4 h. During the addition of water to the mother liquid in step (d), the mixture is preferably agitated. The temperature of the mixture in step (d) is preferably kept from 20° C. to 50° C., more preferably from 25° C. to 45° C., most preferably, from 25° C. to 35° C. According to the specific embodiment, the mixture is held isothermally during the addition of water.

In step (e) the formed solids are separated from the mother liquid by standard methods, e.g. by filtration, sedimentation, decantation or centrifugation. The liquid phase is collected and returned to step (a) or (a) (iii).

In steps d) and e) up to 95% of the compound of formula (I-sym) can be removed as a solid while up to 95% of the compound of formula (I) lost to the mother liquid from step (c) remains in the liquid phase and can then be directly recycled back into the process. This recycling helps to reduce the amount of aromatic solvent used in step (a) and the polar aprotic solvent in step (b).

The process of the invention yield the desired 2-[4-(4-chlorophenoxy)-2-(trifluoro-methyl)phenyl]-1-(1,2,4-triazol-1-yl)propan-2-ol in a purity of at least 98%, in particular at least 98.5%, more particularly at least 99%.

### EXAMPLES

### Example 1

### Preparation of 2-[4-(4-chlorophenoxy)-2-(trifluoromethyl)phenyl]-1-(1,2,4-triazol-1-yl)propan-2-ol (without recycling mother liquid)

243.6 g (0.740 mol) of 2-[4-(4-chlorophenoxy)-2-(trifluoromethyl)phenyl]-2-methyl-oxirane, 56.37 g (0.816 mol) of 1H-[1,2,4]-triazole, 15.75 g (0.393 mol) of NaOH (flakes) and 593 g of DMF were charged to a 1.0 l laboratory vessel at room temperature. The mixture was heated to 115° C. over 12 h for full conversion of the oxirane starting material (yield: 92% in solution for desired isomer).

Afterwards almost the complete DMF (>95%) was removed by vacuum distillation from the reaction mixture. Salts and the remaining DMF were separated from the product by extraction with 727 g toluene and 447 g water at 80° C. Finally 559 g (77%) of the toluene was removed from the product by concentrating the organic phase under vacuum.

DMF for crystallization was added to the product solution in toluene at 85° C. The DMF amount is calculated to give a final concentration of 6% in the pre-crystallization solution. Afterwards the solution in toluene/DMF was cooled to approx. 70° C., seeded with the title product and stirred over 0.5 h. The suspension was slowly cooled down to 10° C. over 8 h for crystallization of the product. The product was separated by centrifugation from the mother liquor and dried in a vacuum oven at 80° C./30 mbar. The dried product was weighed and analyzed for purity. Reaction yields were then calculated and summarized in Table 1 (examples 1.1 to 1.7).

### Example 2

### Preparation of 2-[4-(4-chlorophenoxy)-2-(trifluoromethyl)phenyl]-1-(1,2,4-triazol-1-yl)propan-2-ol (with recycling mother liquid)

The procedure according to Example 1 was repeated.

To 509.8 g of mother liquor plus wash liquor generated from crystallization process above (6.3% of product I and 4.9% of product I-sym), 102 g of water was added over 3 hours while agitating and maintaining mixture temperature at 25° C. The mixture was then filtered to remove the solid, which is primarily (88% concentration after drying) product I-sym. The biphasic filtrate (570 g) was collected to be used as recycle as outlined below.

Starting with 745 g of product 1 (31.0%) and product I-sym (3.13%) in N,N-dimethylformamide, 427.6 g of the polar aprotic solvent was removed by vacuum distillation.

To the remaining distillation heel, the biphasic filtrate above (570 g) was added along with 249.5 g of fresh toluene and 295.0 g of fresh water. The mixture was held at 80° C. for 30 minutes and the phases allowed to separate. 420.5 g of aqueous phase was removed and 992.4 g of organic phase was held for the next step.

Under vacuum, 475.2 g of solvent, primarily toluene, was removed by distillation, leading to a solution containing 51.528% of product 1. N—N-dimethylformamide was then added so that concentration of this polar aprotic solvent was 6.97%.

The solution was then cooled slowly to 75° C., seeded with product, and then cooled to 60° C. over 2 hours, 20° C. over 3 hours and 10° C. over 1 hour and slurry held at temperature for an additional 30 minutes.

Slurry was then filtered and wetcake washed with toluene. The wetcake was dried and analyzed. Reaction yields were then calculated and summarized in Table 2 (examples 2.1 to 2.9).

TABLE 1

| Experiment | |
|---|---|
| Number | Yield, % |
| 1.1 | 73.36 |
| 1.2 | 77.17 |
| 1.3 | 76.57 |
| 1.4 | 79.74 |
| 1.5 | 74.99 |
| 1.6 | 75.53 |
| 1.7 | 78.16 |

TABLE 2

| Experiment | |
|---|---|
| Number | Yield, % |
| 2.1 | 85.42 |
| 2.2 | 85.87 |
| 2.3 | 86.51 |
| 2.4 | 86.26 |
| 2.5 | 86.85 |
| 2.6 | 85.71 |
| 2.7 | 86.12 |
| 2.8 | 85.24 |
| 2.9 | 86.17 |

1. A method for obtaining 2-[4-(4-chlorophenoxy)-2-(trifluoromethyl)phenyl]-1-(1,2,4-triazol-1-yl)propan-2-ol of formula (I) from a mixture containing 2_compound of formula (I) and 2-[4-(4-chlorophenoxy)-2-(trifluoromethyl)phenyl]-1-(1,2,4-triazol-4-yl)propan-2-ol of formula (I-sym) 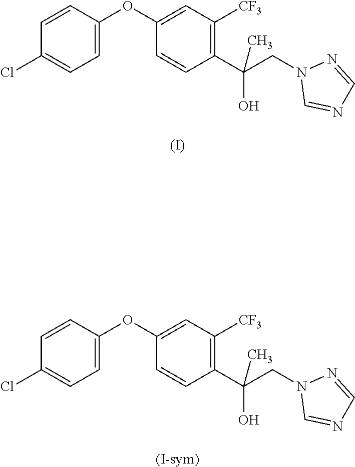 [SMILES: CC(O)(Cn1cncn1)c1ccc(Oc2ccc(Cl)cc2)cc1C(F)(F)F ; "(I)" ; CC(O)(Cn1cncn1)c1ccc(Oc2ccc(Cl)cc2)cc1C(F)(F)F ; "(I-sym)"]
comprising
(a) providing a mixture containing the compounds of formulae (I) and (I-sym) in at least one aromatic solvent, where the mixture contains the compound of formula (I) in a concentration of from 30 to 90% by weight, relative to a total weight of the mixture;
(b) adding to the mixture of step (a) at least one polar aprotic solvent, so that it is contained in an amount of from 1 to 25% by weight, relative to a weight of the solution obtained after the addition of the at least one polar aprotic solvent;
(c) crystallizing the compound of formula (I) from the mixture obtained in step (b);
(d) adding to a mother liquid obtained after crystallization in step (c) water in an amount of from 10 to 40% by weight, relative to a total weight of the mother liquid; and
(e) separating a liquid phase from the mixture obtained in step (d) and returning a separated liquid phase to step (a).

2. The method as claimed in claim 1, wherein step a) comprises:
(i) reacting 2-[4-(4-chlorophenoxy)-2-(trifluoromethyl)phenyl]-2-methyl-oxirane of formula (II) 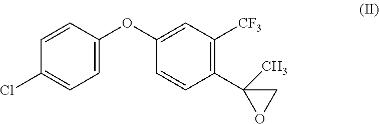 [SMILES: CC1(c2ccc(Oc3ccc(Cl)cc3)cc2C(F)(F)F)CO1]
with 1H-[1,2,4]-triazole in the presence of a base in at least one polar aprotic solvent;
(ii) after completion of the reaction removing at least 90% of the at least one polar aprotic solvent;
(iii) diluting a mixture obtained in step (ii) with at least one aromatic solvent and extracting the diluted mixture with water or an aqueous solution;
(iv) adjusting an amount of the aromatic solvent in the organic phase obtained in step (iii) to such an extent that a resulting mixture contains the compound (I) in a concentration of from 30 to 90% by weight, relative to a total weight of the mixture.

3. The method as claimed in claim 1, where the at least one aromatic solvent used in step (a) is selected from the group consisting of benzene, toluene, xylenes, and mixtures thereof.

4. The method as claimed in claim 1, where the at least one polar aprotic solvent used in step (b) is selected from the group consisting of N,N-dimethylformamide, N,N-dimethylacetamide, dimethylsulfoxide, and N-methylpyrrolidone.

5. The method as claimed in claim 1, where the at least one polar aprotic solvent used in step (b) is N,N-dimethylformamide.

6. The method as claimed in claim 1, wherein water in step (d) is added gradually.

7. The method as claimed in claim 1, wherein a temperature in step (d) is between 2° and 50° C.

8. The method as claimed in claim 3, wherein the at least one aromatic solvent comprises toluene.
